(19) United States
(12) Patent Application Publication
(10) Pub. No.: US 2009/0137526 A1
(43) Pub. Date: May 28, 2009

(54) Purification Method for Biomaterial

(76) Inventors: Carole Jubert, Corvallis, OR (US); John Enrique Mata, Corvallis, OR (US); Scott Bradley Gustafson, Albany, OR (US); John Stephen Dunfield, Corvallis, OR (US)

Correspondence Address: LIFE MICROSYSTEMS INC, 350 NW Elks Dr, Suite 101, CORVALLIS, OR, 97330, US

(21) Appl. No.: 11/944,626

(22) Filed: Nov. 25, 2007

(51) Int. Cl. A61K 31/722; C08B 37/08; A61P 1/00

(52) U.S. Cl. 514/55; 536/20

(57) ABSTRACT

The present disclosure relates to methods for purifying chitin or chitosan, particularly for obtaining medical grade materials suitable for use in biocompatible devices,formulations, or other materials. Chitosan separation and purification methods are taught for obtaining high purity and fractionated bio-compatible medical grade materials. Chemistries and solvent systems are taught for the use in counter current chromatography, liquid/liquid or other microfluidic separation systems.

### FIELD OF THE INVENTION

The present disclosure relates to methods for purifying chitin or chitosan, particularly for obtaining medical grade materials suitable for use in biocompatible devices, formulations, or other materials.

### CROSS REFERENCES CITED

1. L. C. Craig, Journal of Biological Chemistry 150 (1943) 33.
2. L. C. Craig, Journal of Biological Chemistry 155 (1944) 519.
3. N. Kresge, R. D. Simoni, R. L. Hill, Journal of Biological Chemistry 280 (2005) e4.
4. Y. Ito, R. L. Bowman, Science 167 (1970) 281.
5. Y. Wei, T. Zhang, G. Xu, Y. Ito, Journal of Chromatography, A 929 (2001) 169.
6. L. Lei, F. Yang, T. Zhang, P. Tu, L. Wu, Y. Ito, Journal of Chromatography, A 912 (2001) 181.
7. Q. Du, Z. Li, Y. Ito, Journal of Chromatography, A 923 (2001) 271.
8. A. Degenhardt, P. Winterhalter, Special Publication—Royal Society of Chemistry 269 (2001) 143.
9. J. A. Armbruster, R. P. Borris, Q. Jimenez, N. Zamora, G. Tamayo-Castillo, G. H. Harris, Journal of Liquid Chromatography & Related Technologies 24 (2001) 1827.
10. K. A. Alvi, Journal of Liquid Chromatography & Related Technologies 24 (2001) 1765.
11. Y. Ito, Journal of chromatography. A 1065 (2005) 145.
12. Y. Ito, Journal of Chromatography 538 (1991) 3.
13. I. Sutherland, D. Hawes, S. Ignatova, L. Janaway, P. Wood, Journal of Liquid Chromatography & Related Technologies 28 (2005) 1877.
14. Macounova, K., Cabrera, C. R. & Yager, P. Concentration and separation of proteins in microfluidic channels on the basis of transverse IEF. Analytical Chemistry 73, 1627-1633 (2001).
15. Gao, J., Xu, J. D., Locascio, L. E. & Lee, C. S. Integrated microfluidic system enabling protein digestion, peptide separation, and protein identification. Analytical Chemistry 73, 2648-2655 (2001).
16. Weigl, B. H. & Yager, P. Microfluidics—Microfluidic diffusion-based separation and detection. Science 283, 346-347 (1999).
17. Cunningham, D. D. Fluidics and sample handling in clinical chemical analysis. Analytica Chimica Acta 429, 1-18 (2001).
18. A planar microfabricated fluid filter, Brody, J. P., Osborn, T. D., Forster, F. K. and Yager, P., (Proceedings of Transducers '95), Sensors and Actuators A (Physical), A54 (1-3), 704-708, (1996).
19. Optimal design of a microfabricated diffusion-based extraction device, Holl, M. R., Galambos, P., Forster, F. K., Brody, J. P., Afromowitz, M. A., and Yager, P., Proceedings of 1996 ASME Meeting ASME DSC59, 189-195 (1996).
20. Biotechnology at low Reynolds numbers, Brody, J. P., Yager, P., Goldstein, R. E., and Austin, R. H., Biophysical Journal. 71 (6), 3430-3441, (1996).
21. U.S. Pat. No. 7,166,460 Product removal process for use in a biofermentation system
22. U.S. Pat. No. 7,067,640 Cross-linked chiral compounds and methods of making thereof
23. U.S. Pat. No. 6,812,000 Product removal process for use in a biofermentation system
24. U.S. Pat. No. 6,342,592 Chiral compounds, their synthesis and use as a support
25. WO/1996/024842 APPLICATION OF COUNTERCURRENT CHROMATOGRAPHY TO THE DECONVOLUTION OF CHEMICAL LIBRARIES
26. K. L. Mittal, eds Handbook of Microemulsion Science and Technology Promod Kumar ISBN: 978 0824 71979 1

### BACKGROUND OF THE INVENTION

Chitosan (also referred to as poly-(1.fwdarw.4)-.beta.-D-glucosamine) is a biopolymer with many uses in the pharmaceutical, medical device, food and water treatment industries. Chitosan has become an important hemostatic agent since it possesses properties that provide strong bioadhesion to tissue while providing an environment that promotes the formation of coagulum when contacted by defibrinated blood, heparinized blood, or washed red cells. A number of other uses for chitosan have been reported including its use in bone graphs, hemostasis in animals, vascular grafts in dogs, lingual hemostasis in rabbits, and topical hemostasis for diffuse capillary bleeding in animal brains. Although efficacy as external hemostatic bandages have been demonstrated, an important prerequisite for surgical use of chitosan based materials is to reduce the immunological responses induced by the impurities in commercially available chitosan.

Chitosan and its precursor, chitin, are typically prepared from waste shells of crustaceans, particularly decapod crustaceans such as crab, shrimp, crawfish, krill, lobster, squid and prawn. The conventional process for producing chitin and chitosan from crustacean shells involves grinding crustacean shells and treating the ground shells with a dilute base (e.g., sodium hydroxide) and heat to remove protein and lipids (deproteinization). Calcium carbonate is removed by extraction with a dilute acid (e.g., hydrochloric acid) at room temperature (demineralization). Following deproteinization and demineralization, the resulting product is predominantly chitin. An optional decolorization step may be used to bleach the chitin, for example, extraction with ethanol and ether, or bleaching with sodium hypochlorite. Removal of acetyl groups from the chitin polymer (deacetylation) produces chitosan; deacetylation is usually performed by reacting chitin with concentrated sodium hydroxide or potassium hydroxide and heat. The deacetylation process does not remove any contaminants existing in the chitin starting material. Thus, impurity removal for chitosan only occurs during production of the chitin precursor. Chitosan is not a single, definite chemical entity since it varies in composition depending on the crustacean species used for the starting material and the particular preparation method used.

Reducing or substantially removing impurities from chitosan that can cause immunological reactions is critical for chitosan intended for use as a biocompatible and biodegradable material in medical applications. However, purifying chitosan is very difficult since chitosan in solution is a highly viscous material. Producing highly pure, medical grade chitosan via the above-described conventional techniques is very expensive since such techniques typically require costly instrumentation such as autoclaves, ultra filtration, and molecular sieves. The availability of less expensive medical grade chitosan should expand and accelerate its use in biomedical applications.

Counter Current Chromatography

The methods described herein apply counter current techniques to achieve pure chitin and chitosan materials. Counter-current chromatography is a well-known technique that has demonstrated utility for the isolation of phytochemicals. Craig's Counter-Current Distribution (CCD) was a breakthrough in separation science and became very popular in the 1940's [1-3]. CCD devices that filled whole rooms were capable of processing liters of solvents and included up to several hundred partition elements that automated the manual partitioning of samples in separatory funnels. The next generation, counter current chromatography (CCC), was first described by Ito in the early 1970's [4]. Since then, it has been widely used in the field of natural product chemistry to effectively separate a large variety of compounds [5-10]; yet, CCC techniques have been underutilized due to the lack of information available to the beginner. In a recent article [11], Ito describes the basic technical details to quickly learn optimal conditions for CCC separations. As CCC is an all-liquid technique, it benefits from a number of advantages in comparison with the more traditional liquid-solid separation methods, such as column chromatography and HPLC. The main advantage of CCC is that losses by adsorption or denaturation by contact with a solid support are avoided. With a liquid stationary phase, the compounds can diffuse into the volume of the stationary phase, not only its surface, as is the case with classical solid stationary phase. As a result, the loading capability of CCC is much higher than that of HPLC with an equal internal volume.

There are few procedures that can be successfully adapted from laboratory to production scale without difficulties. Preparative HPLC, for example, is not a linear scale-up as the product can become hydrolyzed by or react with the column. CCC can be scaled-up from analytical- to preparative-scale in a completely straightforward manner avoiding these problems [12,13]. Most parameters can be scaled up in proportion to the increase in column volume. The application of counter-current methods to the isolation and purification of chitin and chitosan is an improvement on current techniques that include chemical treatments, enzymatic digestions, precipitations and the use of size exclusion sieves.

### SUMMARY OF THE INVENTION

Disclosed herein are methods for purifying a chitosan starting material (especially food grade chitosan or chitosan powder) that includes at least one pre-existing impurity, particularly protein and/or metal impurities. The disclosed methods reduce the amount of one of more impurity resulting in a more purified chitosan material.

One variant of the method involves contacting a food grade chitosan material or chitosan powder with at least one treatment agent selected from a protein-complexing agent, a metal-chelating agent, and a metal-complexing agent under pH conditions effective for forming a water insoluble chitosan precipitate and at least one water soluble material selected from a water soluble protein complex, a water soluble metal chelate, and a water soluble metal complex. The water insoluble chitosan precipitate and the water soluble material then are separated resulting in a purified chitosan material.

A second variant of the method involves solubilizing the chitosan starting material in an aqueous solution to produce an intermediate chitosan material. The intermediate chitosan material is contacted with at least one treatment agent selected from a deproteinization agent and a demetallization agent under pH conditions effective for forming a water insoluble chitosan precipitate and at least one water-soluble material that includes the pre-existing impurity. The water insoluble chitosan precipitate and the water-soluble material then are separated resulting in a purified chitosan material.

The disclosed methods will become more apparent from the following detailed description of several embodiments.

### BRIEF DESCRIPTION OF THE DRAWINGS

FIG. 1: Illustrates the purification of the silylated chitin by liquid-liquid extraction. The chitin is then de-silylated, followed by de-acetylation to obtain pure chitosan.

### DETAILED DESCRIPTION OF SEVERAL EMBODIMENTS

For ease of understanding, the following terms used herein are described below in more detail:

“Protein” is inclusive of one or more proteins and encompasses proteins, polypeptides, peptides, fragments thereof, and conjugated proteins.

“Metal” is inclusive of one or more metals and encompasses metallic salts, metal oxides, metal hydrides, metal ions, and elemental metal. The metal can be any type of metal such as, for example, an alkali metal, an alkaline-earth metal, a transition metal, a rare-earth metal, or mixtures thereof.

“Endotoxin” is inclusive of heat stable polysaccharide like toxins bound to a bacterial cell. The term specifically refers to lipopolysaccharide (LPS) of the outer membrane of gram-negative bacteria. There are three parts to the molecule, the Lipid A (six fatty acid chains linked to two glucosamine residues), the core oligosaccharide (branched chain of ten sugars) and a variable length polysaccharide side chain (up to 40 sugar units in smooth forms).

The above term descriptions are provided solely to aid the reader, and should not be construed to have a scope less than that understood by a person of ordinary skill in the art or as limiting the scope of the appended claims.

One feature of the methods disclosed herein is that commercially available chitosan containing higher levels of impurities can be used as the starting material for further purification. In conventional methods, the impurity level in a particular chitosan product is controlled in one aspect during production of the chitin precursor. In a second aspect, impurity levels can be controlled during the chemical transformation of chitin precursor into a chitosan product. Food grade chitosan is one example of a less-expensive, more-contaminated chitosan that can be used as a starting material. Food grade chitosan is significantly less expensive than commercially available medical grade chitosan. Food grade chitosan includes a level of impurities that is sufficiently low for safe human consumption, but too high for medical use as a biocompatible material. Typically, a food grade chitosan material will have a protein impurity level of less than about 1.2 weight percent, more particularly about 0.4 to about 0.8 weight percent, and a metal impurity level of about 70 to about 80 ppm (with a heavy metal content of less than about 7.5 ppm), and will include pigment. The chitosan starting material can be in any physical form such as a powder or a mixture of small fibers and particles.

Another feature of the disclosed methods is their cost effectiveness. The disclosed methods utilize less expensive countercurrent chromatographic techniques for generating the purified chitosan rather than the more expensive autoclaving, molecular sieves and/or ultra filtration. Thus, chitosan purified according to the detailed methods should be considerably less expensive to produce compared to the presently available medical grade chitosan.

Although the more-contaminated chitosan starting material may be subjected to deproteinization and/or demetallization treatments prior to purification using methods disclosed herein, it is not a required. In other words, the counter current chromatographic techniques described herein will substantially purify the chitosan (or chitin) material away from the contaminating proteins, endotoxins, and metals.

Following purification of chitosan using counter current chromatography techniques described herein, the chitosan or chitin material can be precipitated out of the mobile phase eluant. The water insoluble chitosan and water-soluble supernatant may be separated by any known technique such as centrifugation, filtration or a combination thereof. Filtration, for example, can be performed with a filter material such as MIRACLOTH (commercially available from CalBiochem). Separation of the water insoluble chitosan and water-soluble supernatant may be performed prior to any subsequent purification of the chitosan. Alternatively, subsequent purification of the chitosan may be performed using the mixture of the water insoluble chitosan and the water-soluble supernatant.

The disclosed methods envision a possible need for demetallization agent(s) for mixing with the chitosan material may be any substance such as a metal-chelating or metal-complexing agent that can remove at least a portion of the metal present in the chitosan material. The demetallization agent can be mixed with the chitosan material under basic pH conditions to avoid the formation of a chitosan-metal chelate conjugate or the demetallization agent may be added to the stationary phase to bind metals as the chitosan containing mobile phase passes through the apparatus.

Illustrative metal-chelating agents include the following organic acids and their isomers and salts: ethylenediaminetetraacetic acid (EDTA), nitrilotriacetic acid (NTA), diethylenetriaminepentaacetic acid (DTPA), ethyleneglycol-bis(2-amino-ethylether)N,N,N′N′-tetraacetic acid (EGTA), butylenediaminetetraacetic acid, (1,2-cyclohexylenedinitrilo-)tetraacetic acid (CyDTA), ethylenediaminetetrapropionic acid, (hydroxyethyl)ethylenediaminetriacetic acid (HEDTA), N,N,N′,N′-ethylenediaminetetra(methylenephosphonic) acid (EDTMP), triethylenetetraminehexaacetic acid (TTHA), 1,3-diamino-2-hydroxypropane-N,N,N′,N′-tetraacetic acid (DHPTA), methyliminodiacetic acid, propylenediaminetetraacetic acid, 1,5,9-triazacyclododecane-N,N′,N″-tris(methylenephosphonic acid) (DOTRP), 1,4,7,10-tetraazacyclododecane-N,N′,N″,N′″-tetrakis(methylenepho-sphonic acid) (DOTP), nitrilotris(methylene) triphosphonic acid, diethylenetriaminepenta(methylenephosphonic acid) (DETAP), aminotri(methylenephosphonic acid), 1-hydroxyethylene-1,1-diphosphonic acid, bis(hexamethylene)triamine phosphonic acid, 1,4,7-triazacyclononane-N,N′,N″-tris(methylenephosphonic acid (NOTP), 2-phosphonobutane-1,2,4-tri-carboxylic acid, citric acid, tartaric acid, gluconic acid, saccharic acid, glyceric acid, oxalic acid, phthalic acid, maleic acid, mandelic acid, malonic acid, lactic acid, salicylic acid, 5-sulfosalicylic acid, catechol, gallic acid, propyl gallate, pyrogallol, 8-hydroxyquinoline, and cysteine.

Removing insoluble particles, such as residual chitin, protein, polysaccharides, and polysaccharide conjugates, from the chitosan starting material is an optional initial step. This step can be accomplished by adding a sufficient amount of a dilute aqueous acid to the chitosan starting material to solubilize the chitosan. The pH of the resulting solution should be about 2.0 to about 4.5. Illustrative solubilizing agents for solubilizing chitosan include organic acids having 1 to 10 carbon atoms, particularly 2 to 7 carbon atoms, such as formic acid, acetic acid, butyric acid, glutamic acid, tartaric acid, succinic acid, lactic acid, ascorbic acid, propionic acid, and adipic acid, and mineral acids such as hydrochloric acid. Acetic acid, glutamic acid, tartaric acid, ascorbic acid and lactic acid are especially suitable. It has been found that in certain instances utilization of an initial solubilization coupled with deproteinization may result in a purified chitosan with a lower amount of residual protein compared to using deproteinization without an initial solubilization step.

A further optional purification treatment agent is a reducing agent for enhancing the water solubilization of protein impurities by dissociating any disulfide bonds present in the protein. The amount of reducing agent mixed with the chitosan material is not critical and may range, for example, from about 1 mM to about 5 mM, depending on the level of protein contamination. Examples of suitable reducing agents include sulfhydryl reducing agents such as, for example, dithiothreitol (DTT), dithioerythritol (DTE) and mercaptoethanol.

The purified chitosan made by the methods disclosed herein is useful in numerous pharmaceutical, medical device, food and water treatment applications. The purified, biocompatible chitosan may be used as a delivery vehicle for a therapeutically active agent, in a synthetic bone graft material (e.g., a mixture of chitosan and hydroxyapatite), in spinal fusions, and in various wound-healing applications such as hemostatic bandages or sponges. For example, U.S. Pat. Nos. 5,836,970; 5,420,197; 5,395,305; 4,956,350; 4,699,135; 4,659,700; 4,651,725; 4,614,794; 4,572,906; 4,570,629; 4,532,134; 4,394,373; 3,903,268; 3,632,754; and 6,056,970 describe various wound dressings, hemostatic sponges or hemostatic bandages made from chitosan. The purified chitosan may also be used for repair and regeneration of cartilage and other tissues as described, for example, in U.S. Published patent application No. 20020082220. The purified chitosan may be used in various physical forms such as fibers, films, gels or powders. For example, chitosan fibers may be made using wet spinning techniques as described in U.S. Pat. Nos. 5,897,821 and 5,836,970. The purified chitosan may also be used to make chitosan derivatives such as chitosan lipoate, chitosan poly(ethylene glycol), chitosan oligosaccharide lactate, chitosan neutralized with pyrrolidone carboxylic acid, carboxymethyl sodium salt of chitosan, chitosan neutralized with glutamic acid, and N,O-carboxymethyl chitosan.

The counter current extraction is carried out in accordance with any manner and with any extraction equipment in any period of time such that the extraction is achieved. For example, the extraction may be carried out in a multistage extraction column in counter current manner. Further distillation of extractant may be carried out in accordance with any manner at any conditions such that the distillation is achieved. Other methods may be employed to allow chitin and chitosan isolation similar to that achieved with counter current extraction. Another embodiment of the invention includes the use of microfluidic properties (18-20) applied to the purification process in order to achieve sufficient extraction and isolation to provide substantially pure chitin or chitosan.

The invention will be more readily understood by reference to the following examples. There are, of course, many other forms of this invention which will become obvious to one skilled in the art, once the invention has been fully disclosed, and it will accordingly be recognized that these examples are given for the purpose of illustration only, and are not to be construed as limiting the scope of this invention in any way.

### EXAMPLE 1

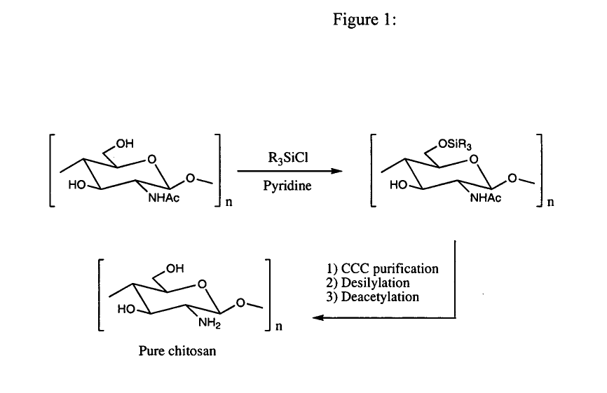

Silylated (such as trimethyl, triphenyl silyl) derivatives of chitin are prepared prior to purification by liquid-liquid extraction. These new derivatives of chitin are found to be soluble in most organic solvents, such as dioxan, tetrahydrofuran, chloroform, toluene, acetone and are suitable for use in the method described herein. A two-phase solvent system made of such organic solvent and an aqueous phase, where the K values of the silylated chitin are in the proper range, is then used for the purification of the silylated chitin by liquid-liquid extraction. The chitin is then de-silylated, followed by de-acetylation to obtain pure chitosan. Refer to FIG. 1.

### EXAMPLE 2

Chitosan can be purified by using an aqueous two-phase solvent system. Aqueous two-phase systems (ATPS) consist of two immiscible phases formed when certain water-soluble polymers (e.g., polyethylene glycol (PEG), dextran) are combined with one another or with certain inorganic salts (e.g., (NH₄)₂SO₄, K₃PO₄) in specific concentration. Success with aqueous two-phase systems depends on the ability to manipulate phase composition so as to obtain appropriate partition coefficients and selectivity for the material of interest. The aqueous polymer phase system is prepared by dissolving the polymer/polymer systems (examples given below) in distilled water. To achieve efficient separation of chitosan, it is essential to optimize the partition coefficient of chitosan by selecting a proper pH of the polymer phase systems. The pH of the system is adjusted below 6.5 by choosing the proper ratio between the mono- and dibasic salts. An appropriate ratio, which can yield approximately equal volumes of upper and lower phases is determined prior to purification. The partition coefficients are determined by the measurement of the refractive index.

| | |
|---|---|
| Component 1 | Component 2 |

Examples of Polymer/polymer Systems are listed in the following list:

| | |
|---|---|
| Polyethylene glycol | Dextran |
| | Ficoll |
| | Polyvinyl pyrrolidone |
| | Polyvinyl alcohol |
| | Hydroxypropyl starch |
| Polypropylene glycol | Dextran |
| | Hydroxypropyl dextran |
| | Polyvinyl pyrrolidone |
| Polyvinyl alcohol | Dextran |
| | Hydroxypropyl dextran |
| Polyvinyl pyrrolidone | Dextran |
| | Maltodextrin |
| Methyl cellulose | Dextran |
| | Hydroxypropyl dextran |
| Ethylhydroxyethyl cellulose | Dextran |

Examples of Polymer/salt Systems are listed in the following list:

| | |
|---|---|
| Polyethylene glycol | Potassium phosphate |
| | Sodium sulfate |
| | Magnesium sulfate |
| | Ammonium sulfate |
| | Sodium citrate |
| Polypropylene glycol | Potassium phosphate |
| Methoxypolyethylene glycol | Potassium phosphate |
| Polyvinyl pyrrolidone | Potassium phosphate |

Several ways to manipulate system composition so as to give phases with appreciably different physical properties will be investigated; e.g., (1) choice of polymers, polymer concentration, polymer molecular weight; (2) choice of salt(s) and salt concentration; and (3) chemical modification of one of the polymers by attaching a ligand for which receptors exist on chitosan.

### EXAMPLE 3

In order to improve the above separation, a pH-peak focusing CCC technique can be applied to the conventional CCC method described in EXAMPLE 2. pH-zone-refining CCC is generally employed as a large-scale preparative technique for separating ionizable analytes. pH-zone-refining CCC separates organic acids and bases into a succession of highly concentrated rectangular peaks that elute according to their pKₐ's and hydrophobicities. This purification technique increases the sample capacity at least 10-fold over conventional CCC and produces highly concentrated purified fractions. The pH-zone-refining CCC technology uses a retainer acid (or base) in the stationary phase and an eluent base (or acid) in the mobile phase. This combination, acting in concert with the planetary motion of the CCC column, causes multiple solute transfers.

### EXAMPLE 4

EXAMPLES 2 AND 3 above have utility for the analysis of chitosan collected from biological samples after application of chitosan based materials and therefore the methods described are useful in the analysis of degradation, bioabsorption or chemical transformation of chitosan.

### EXAMPLE 5

Examples 1, 2 and 3 above have utility for the analysis of chitin or chitosan for the purpose of quality control of purified or modified chitins or chitosan

### EXAMPLE 6

Example 5 above has utility for the analysis of chitin or chitosan based medical devices for the purpose of quality control

### EXAMPLE 7

Pre or post CCC processing of chitin or chitosan with microfluidic separation methods (18-20) allow for faster and/or more specific separation.

### EXAMPLE 8

Pre or concurrent CCC processing of at least two immiscible phases to create unstable microemulsions (26) and/or liposome mixtures results in a faster and higher yield separation process. Microemulsions or liposome like phases are created by isolated centrifuge, ejection of microdroplets less than about 20 um of one phase into the other immiscible phase, or injection of a high velocity gas phase into the two immiscible liquid phases. Creation of microemulsions or liposomes can occur prior to loading the CCC but the preferred method is to create them in-situ for purposes of precise control of the size distribution and thermodynamic properties in a continuous flow.

What is claimed is:

1) A method for purifying a chitosan material that includes at least one pre-existing impurity, the method comprising: contacting the chitosan material with at least one solvent under pH conditions effective for forming a soluble chitosan solution: and separating the chitosan from the impurities contained within by application of liquid/liquid chromatography or microfluidic operations resulting in a purified chitosan material that includes a lower amount of impurity, relative to the initial chitosan material.

2) The method of claim 1, wherein countercurrent chromatography is the purification process.

3) The method of claim 1, wherein microfluidic and microdroplet properties are applied to the purification process

4) The method of claim 1, wherein the pre-existing impurity is selected from protein, endotoxin, or metal

5) The method of claim 1, wherein the chitosan material has at least one pre-existing impurity selected from protein in an amount of less than about 1.2 weight percent, metal in an amount of about 70 to about 80 ppm and heavy metal in an amount of less than about 7.5 ppm.

6) The method of claim 1, wherein the chitosan material is contacted with a metal-chelating agent.

7) The method of claim 1, wherein the contacting of the chitosan material with the treatment agent occurs under controlled pH conditions.

8) The method of claim 1, further comprising contacting the chitosan with a sulfhydryl reducing agent.

9) A method of claim 1, further comprising producing an ultra pure medical grade chitosan material from purified chitosan.

10) The method of claim 1, wherein the amount of protein in the purified chitosan material is less than about 0.1 weight percent or the amount of metal in the purified chitosan material is less than about 5 ppm.

11) The method of claim 1, further comprising forming the purified chitosan material into a hemostatic article or for forming a biomaterial for tissue treatment or reconstruction or for enteric use as a gastrointestinal protectant.

12) A method for purifying a raw chitin material that includes at least one pre-existing impurity selected from protein, endotoxin or metal, the method comprising: contacting the chitin material with at least one solvent under pH conditions effective for forming a soluble chitin solution: and separating the chitin from the impurities contained within by application of liquid/liquid chromatography or microfluidic operations resulting in a purified chitin material that includes a lower amount of at least the protein or metal or endotoxin impurity, relative to the raw chitin material.

13) The method of claim 12, wherein countercurrent chromatography is the purification process.

14) The method of claim 12, wherein microfluidic and microdroplet properties are applied to the purification process

15) A method of claim 12, further comprising producing an ultra pure medical grade chitosan material from purified chitin.

16) The method of claim 12, wherein the raw chitin material has at least one pre-existing impurity selected from protein in an amount of less than about 1.2 weight percent, metal in an amount of about 70 to about 80 ppm and heavy metal in an amount of less than about 7.5 ppm.

17) The method of claim 12, wherein the raw chitin material is contacted with a metal-chelating agent.

18) The method of claim 12, wherein the contacting of the raw chitin material with the treatment agent occurs under controlled pH conditions.

19) The method of claim 12, further comprising contacting the raw chitin material with a sulfhydryl reducing agent.

20) The method of claim 12, wherein the amount of protein in the purified chitin material is less than about 0.1 weight percent or the amount of metal in the purified chitin material is less than about 5 ppm.

21) The method of claim 12, further comprising forming the purified chitin material into a hemostatic article or for forming or creation of a biomaterial for tissue treatment or reconstruction or enteric use as a gastrointestinal protectant.

22) The method of claim 1, wherein the source of chitosan is collected from biological tissue for the purpose of analysis of degradation, bioabsorption, or contamination.

23) The method of claim 12, wherein the source of chitin is collected from biological tissue for the purpose of analysis of degradation, bioabsorption, or contamination.

24) A method for further separating chitosan or chitin material by range of molecular weights.

25) The method of claim 24, further comprising forming a biomaterial of specific molecular weight into a hemostatic article or for forming or creation of a biomaterial for tissue treatment or reconstruction or enteric use as a gastrointestinal protectant.
